(19) United States
(12) Patent Application Publication
(10) Pub. No.: US 2016/0326073 A1
(43) Pub. Date: Nov. 10, 2016

(54) PROCESS FOR PRODUCING NEOPENTYL GLYCOL

(71) Applicant: OXEA GMBH, Oberhausen (DE)

(72) Inventors: Matthias Eisenacher, Wesel (DE); Kurt Schalapski, Oberhausen (DE); Heinz Strutz, Moers (DE)

(21) Appl. No.: 15/037,346

(22) PCT Filed: Jan. 26, 2015
(86) PCT No.: PCT/EP2015/051466

§ 371 Date: May 18, 2016

(30) Foreign Application Priority Data
Jan. 28, 2014 (DE) 102014100996.7

(51) Int. Cl. C07C 29/141; C07C 45/75; B01J 23/889; C07C 29/84

(52) U.S. Cl. CPC C07C 29/141; C07C 29/84; C07C 45/75; B01J 23/8892

(57) ABSTRACT

The present application describes a method for the synthesis of neopentylglycol starting from isobutyraldehyde and formaldehyde which are reacted in the presence of an alkaline catalyst, followed by separation of the volatile components until a water content of less than 5% is obtained. The distillation bottom product is then hydrogenated.

### CLAIM FOR PRIORITY

This application is a national phase application of PCT/EP2015/051466 FILED Jan. 26, 2015 which was based on application DE 10 2014 100 996.7 FILED Jan. 28, 2014. The priorities of PCT/EP2015/051466 and DE 10 2014 100 996.7 are hereby claimed and their disclosures incorporated herein by reference.

### TECHNICAL FIELD

The invention relates to a process for producing neopentylglycol.

### BACKGROUND

Neopentylglycol (=2,2-dimethyl-1,3-propanediol) is an important compound of the industrial chemistry, mainly as raw material for the production of saturated polyester resins for powder coatings as well as for glass fiber reinforced plastics.

For this reason there is a constant search for new, improved methods of producing neopentylglycol.

### SUMMARY OF INVENTION

It is therefore an object to provide a new method of producing neopentylglycol. This object is achieved by a method according to claim 1 of the present invention. Accordingly, a method is provided comprising the steps of:

a) reacting isobutyraldehyde with formaldehyde in the presence of an alkaline catalyst, wherein the isobutyraldehyde is present in excess;

b) purification by distillation of the reaction mixture obtained from step a) so that a bottom product containing 5 wt. % of water is obtained; and

c) hydrogenating the bottom product into neopentylglycol.

Surprisingly it has been found that by a reaction in an alkaline region, in which the isobutyraldehyde is present in excess, in the subsequent purification by distillation a bottom product can be obtained which contains 5% or less water and is moreover completely free of the catalyst of the previous aldol reaction in most applications. Such a bottom product can subsequently be hydrogenated so that not only hydroxypivalin aldehyde is reacted selectively into neopentylglycol, but also high-boiling impurities can be reacted into neopentylglycol highly selectively.

### DETAILED DESCRIPTION

The individual steps are explained in more detail below:

Step a) Reaction of isobutyraldehyde with formaldehyde

This reaction is preferably carried out at a temperature of ≧40° C. to ≦100° C. and can be carried out continously or discontinously.

In the vast majority of applications formaldehyde is used in the form of an aqueous solution of formaldehyde.

Herein, the isobutyraldehyde is present in excess with respect to the formaldehyde present, preferably a ratio of ≧1.01:1 (mol isobutyraldehyde to mol formaldehyde), more preferably ≧1.03:1, further preferably ≧1.05:1 to ≦1.2:1 and most preferably ≧1.1:1 to ≦1.15:1.

Step a) is carried out in the presence of an alkaline catalyst, wherein the alkaline catalyst is preferably used in a molar ratio of ≧0.01 to ≦0.1 with respect to isobutyraldehyde.

Preferably, the alkaline catalyst comprises trimethylamine and/or an aqueous alkaline solution, preferably a sodium and/or potassium hydroxide solution.

If the alkaline catalyst comprises an aqueous alkaline solution, preferably a step al) is carried out after step a):

a1) separation of the aqueous phase prior to step b).

Step b) Purification by distillation

Step b) is preferably carried out in a thin film evaporator. This preferably comprises an attached column with 10 to 30 floors. Moreover, temperatures of ≧170° C. to ≦200° C. are preferred.

Since in step a) isobutyraldehyde is present in excess, a two-phase system consisting of an organic phase which mainly contains isobutyraldehyde and an aqueous phase is obtained in the distillate during the distillation process; this allows to return the isobutyraldehyde without further purification to step a).

According to a preferred embodiment of the invention, thus, the method comprises a step b1) which is carried out after step b):

b1) returning the organic isobutyraldehyde containing phase separated in step b) for a repeated reaction according to step a).

Step b) is preferably carried out so that the bottom product contains ≦3 wt. %, preferably ≦2 wt. % of water.

According to a preferred embodiment of the present invention the organic phase is supplied at least on the 6th floor of the attached column. It has been found that in this way a particular good separation efficiency is possible.

Step c) Hydrogenation

Preferably, step c) is carried out immediately after step b), that is, according to a preferred embodiment of the invention no further purification of the distillation bottom product takes place between steps b) and c).

Preferably step c) is carried out at a pressure of ≧6 MPa to ≦20 MPa hydrogen, more preferably ≧8 MPa to ≦18 MPa.

Preferably step c) is carried out at a temperature of ≧100° C. to ≦220° C., this has been proven in practice.

Step c) can be carried out in a single stage reactor. However, a two or more stages hydrogenation in a multizone reactor is particularly preferred. In this case, the pressure conditions (see above) then respectively are the pressure conditions over the entire reactor.

If a two-stage hydrogenation is selected, this will preferably be carried out such that in the zone of the reactor, which is first reached by the material to be hydrogenated it is operated at ≧100 to ≦140° C. and a ratio V/Vh of ≧0.7 to ≦1.0 h⁻¹ (with respect only to the first zone of the reactor) and in the subsequent zone of the reactor it is operated at ≧150 to ≦220° C. and a ratio V/Vh of ≧0.2 to ≦0.8 h⁻¹ (with respect only to the second zone of the reactor). This has been proven in practice.

The catalyst preferably comprises a catalyst based on nickel and/or copper chromite, preferably with manganese and/or barium doping. This has been proven in practice.

The product then, depending on the application and the concrete field of application of the method can subjected to further purification steps such as distillation according to the specifications etc. These also represent preferred embodiments of the present invention.

The components to be used according to the invention mentioned above and claimed and described in the exemplary embodiments are not subjected to any exceptional conditions with respect to their size, shape, substrate selection and technical conception, so that the selection criteria well-known in the field of application can be applied without any restriction.

Further details, features and advantages of the subject matter of the invention are obvious from the dependent claims and from the following description of an example, which is to be understood as purely illustrative.

### EXAMPLE

### Production of the Hydrogenation Catalyst Used

2.8 kg of copper nitrate trihydrate, 400 g of manganese nitrate (50% solution in dilute nitric acid) and 150 g of barium nitrate are dissolved in 20 l of destilled H₂O at 55° C. Separately 2.6 kg of ammonium dichromate are dissolved in 12 l of water and 4 l of 25% ammonia solution.

Thereafter, the ammonium dichromate solution is slowly dripped into the copper nitrate solution. Thus, a red-brown solid is precipitated. In order to complete the precipitation process subsequently the product is stirred for one hour and cooled down to room temperature. Subsequently the solid is filtered off. Then the solid is dried at 110° C. in a compartment dryer. The dried solid is calcined at 350° C. for four hours at a heating rate of 2° C./min.

After the calcination of the solid and recooling the solid it is stirred by use of 20 l 10% acetic acid. Then the solid is washed with water until acid-free and dried again at 110° C. and annealed at 350° C. (heating rate 2° C./min).

Subsequently, the solid can be used as a catalyst.

With respect to the metals the catalyst had the following composition based on the proportions of copper, chromium, manganese and barium:

47.5% copper, 46.5% chromium, 4.0% manganese, 2.0% barium.

Execution of the Process According to the Invention:

3996 g of isobutyraldehyde (97.4%) and 3034 g of formalin (49% aqueous solution) are added to an autoclave and heated to 45° C. Then 151 g of trimethylamine (40% aqueous solution) are pumped in. Once the addition is completed, the reaction mixture is heated to 90° C. and left at this temperature for one hour. Then the product is discharged. The composition of the product is:

| Substance | Amount/g |
|---|---|
| Trimethylamine | 60 |
| Isobutyraldehyde | 225 |
| Methanol | 10 |
| Hydroxypivalinaldehyde | 4931 |
| Neopentylglycol monoisobutyrate | 26 |
| Neopentylglycole | 29 |
| Hydroxypivalic acid neopentylglycolester | 151 |
| Water | 1720 |
| Other organic compunds | 29 |

This mixture is then processed by distillation in a thin film evaporator with attached column. In this case, the mixture is fed to floor 20 of a 26 floor packed-bed column. The thin film evaporator is operated at 170° C. In this process a two-phase distillation top product and a distillation bottom product are obtained. The organic phase of the distillation top product has the following composition:

| Substance | Amount/g |
|---|---|
| Trimethylamine | 23 |
| Isobutyraldehyde | 185 |
| Hydroxypivalinaldehyde | 102 |
| Water | 11 |
| Other organic compounds | 37 |

This organic phase can subsequently be used again in the aldolization process without further processing.

The aqueous phase of the distillation top product has the following composition:

| Substance | Amount/g |
|---|---|
| Trimethylamine | 37 |
| Isobutyraldehyde | 25 |
| Hydroxypivalinaldehyde | 8 |
| Water | 1598 |
| Other organic compounds | 23 |

This aqueous phase is then discarded.

The distillation bottom product has the following composition:

| Substance | Amount/g |
|---|---|
| Isobutyraldehyde | 15 |
| Hydroxypivalinaldehyde | 4776 |
| Cyclic acetal from neopentylglycol and | 16 |
| hydroxypivalinaldehyde | |
| Neopentylglycol monoisobutyrate | 25 |
| Neopentylglycol | 23 |
| Hydroxypivalic acid neopentylglycolester | 212 |
| Water | 111 |
| Other organic compounds | 88 |

The distillation bottom product is then hydrogenated, this is done as follows:

The above mentioned catalyst is mixed with 3% graphite and tableted. The resulting 5×5 mm tablets are placed in a tubular reactor with a volume of 1.3 liter. Here, the reactor is equipped so that the lower 0.3 liter of the catalyst bed can be heated separately and the upper 1.0 liter of the catalyst bed can also be heated separately.

For the catalyst activation both catalyst beds are each heated to the same temperature. The catalyst is activated as follows:

Heating rate 20° C./h to 180° C.,

Nitrogen 1000 nl/h

Hydrogen 20 nl/h

Duration 12 hours

Nitrogen 1000 nl/h

Hydrogen 60 nl/h

Duration 6 hours

Nitrogen 1000 nl/h

Hydrogen 120 nl/h

Duration 6 hours

Subsequently, the lower bed is heated to 130° C. and the upper bed is heated to 170° C., a hydrogen pressure of 8 MPa is applied and 300 ml/h of the distillation bottom product from Example 2 are conveyed into the reactor from below. The product thus obtained has the following composition:

| Substance | Amount/g |
|---|---|
| Isobutanol | 25 |
| Neopentylglycol | 5000 |
| Hydroxypivalic acid neopentylglycolester | 21 |
| Water | 111 |
| Other organic Compounds | 108 |

The product can then be purified by known methods.

The individual combinations of the ingredients and the features of the embodiments mentioned above are exemplary. The person skilled in the art will recognize that variations and modifications from the embodiments described herein and other embodiments may also occur without departing from the spirit and scope of the invention. Accordingly, the above description is to be considered exemplary rather than limiting. The word “include” or “comprise” used in the claims does not exclude other elements or steps. The indefinite article “a” does not exclude the importance of a plural. The mere fact that certain measures are recited in mutually different claims, does not indicate that a combination of these measures can not be used advantageously. The scope of the invention is defined in the following claims and its equivalents.

1. Method for the synthesis of neopentylglycol, comprising the steps of:
a) reacting isobutyraldehyde with formaldehyde in the presence of an alkaline catalyst, wherein the isobutyraldehyde is present in excess;
b) purifying the reaction mixture from a) by distillation, so that a bottom product is obtained which contains less than 5 wt. % of water;
c) hydrogenating the bottom product into neopentylglycol;

2. Method according to claim 1, wherein the ratio of isobutyraldehyde to formaldehyde is ≧1.01:1 (mol isobutyraldehyde to mol formaldehyde).

3. Method according to claim 1, wherein the alkaline catalyst is preferably used in a molar ratio of ≧0.01 to ≦0.1 with respect to isobutyraldehyde.

4. Method according to claim 1, wherein the alkaline catalyst comprises trimethylamine and/or alkali hydroxide solution.

5. Method according to claim 1, wherein step b) is carried out within a thin film evaporator.

6. Method according to claim 1, further comprising the step b1) which is carried out after step b):
b1) returning the organic isobutyraldehyde containing phase separated in step b) for a repeated reaction according to step a).

7. Method according to claim 1, wherein between steps b) and c) no further purification of the distillation bottom product occurs.

8. Method according to claim 1, wherein step c) is carried out at a pressure of ≧6 MPa to ≦20 MPa hydrogen.

9. Method according to claim 1, wherein step c) is carried out as a two or multi-stage hydrogenation process in a multizone reactor.

10. Method according to claim 1, wherein step c) comprises a catalyst based on nickel and/or copper chromite.

11. Method according to claim 2, wherein the alkaline catalyst is preferably used in a molar ratio of ≧0.01 to ≦0.1 with respect to isobutyraldehyde.

12. Method according to claim 2, wherein the alkaline catalyst comprises trimethylamine and/or alkali hydroxide solution.

13. Method according to claim 3, wherein the alkaline catalyst comprises trimethylamine and/or alkali hydroxide solution.

14. Method according to claim 2, wherein step b) is carried out within a thin film evaporator.

15. Method according to claim 3, wherein step b) is carried out within a thin film evaporator.

16. Method according to claim 4, wherein step b) is carried out within a thin film evaporator.

17. Method according to claim 2, further comprising the step b1) which is carried out after step b):
b1) returning the organic isobutyraldehyde containing phase separated in step b) for a repeated reaction according to step a).

18. Method according to claim 3, further comprising the step b1) which is carried out after step b):
b1) returning the organic isobutyraldehyde containing phase separated in step b) for a repeated reaction according to step a).

19. Method according to claim 4, further comprising the step b1) which is carried out after step b):
b1) returning the organic isobutyraldehyde containing phase separated in step b) for a repeated reaction according to step a).

20. Method according to claims 5, further comprising the step b1) which is carried out after step b):
b1) returning the organic isobutyraldehyde containing phase separated in step b) for a repeated reaction according to step a).
